(19) United States
(12) Patent Application Publication
(10) Pub. No.: US 2021/0275426 A1
(43) Pub. Date: Sep. 9, 2021

(54) COSMETIC COMPOSITION COMPRISING ANIONIC, NON-IONIC AND AMPHOTERIC SURFACTANTS, AT LEAST TWO ORGANOSILANES AND ASSOCIATIVE CATIONIC POLYMERS, COSMETIC TREATMENT PROCESS AND USE

(71) Applicant: L'OREAL, Paris (FR)

(72) Inventors: Adrien BENAZZOUZ, SAINT-OUEN (FR); Laura FENELON, SAINT-OUEN (FR)

(21) Appl. No.: 17/252,487

(22) PCT Filed: Jun. 13, 2019
(86) PCT No.: PCT/EP2019/065459

§ 371 Date: Dec. 15, 2020

(30) Foreign Application Priority Data
Jun. 28, 2018 (FR) 1855838

(51) Int. Cl. A61K 8/73; A61K 8/46; A61K 8/60; A61K 8/58; A61K 8/44; A61Q 5/12; A61Q 5/02

(52) U.S. Cl. CPC A61K 8/731; A61K 8/463; A61K 8/604; A61K 8/585; A61K 2800/548; A61Q 5/12; A61Q 5/02; A61K 2800/5426; A61K 8/442

(57) ABSTRACT

The present invention relates to a cosmetic composition, in particular a cosmetic hair composition, comprising anionic surfactants, amphoteric surfactants, non-ionic surfactants, at least two organosilanes that are different from one another, and associative cationic polymers. The invention also relates to a process for the cosmetic treatment of keratin materials, in particular the hair, using the composition above, and also to the use thereof in particular for conferring conditioning properties, in particular volume and body properties, on the hair.

The present invention relates to a cosmetic composition, in particular a cosmetic hair composition, most particularly for cleansing and/or conditioning the hair, comprising a particular combination of anionic, non-ionic and amphoteric surfactants, and also organosilanes and one or more associative cationic polymers. The invention also relates to a process for the cosmetic treatment of keratin materials using said composition, and also to the use of said composition for the cosmetic treatment of keratin materials, in particular for conferring conditioning properties on said keratin materials.

Hair can generally be damaged and embrittled by the action of external atmospheric agents such as light and bad weather, and/or by mechanical or chemical treatments, such as brushing, combing, dyeing, bleaching, permanent-waving and/or relaxing. To remedy these drawbacks, it is now common practice to have recourse to haircare compositions which make it possible to condition the hair after these treatments to give it in particular sheen, softness, suppleness, lightness, a natural feel and disentangling properties. These hair compositions may be, for example, conditioning shampoos, compositions to be applied before shampooing (pre-shampoo) or compositions to be applied after shampooing (conditioner), and may be in the form of gels, hair lotions or creams of varying thickness.

Hair compositions, in particular intended for hair cleansing and conditioning, comprising anionic surfactants, non-ionic surfactants, amphoteric surfactants and particular cationic and amphoteric polymers are thus known, in particular through applications FR 3040300 and FR 3040302. These compositions are very stable in terms of viscosity and visual appearance, and make it possible to improve the cosmetic properties conferred on the hair, in particular the disentangling, the suppleness and the feel of said hair.

Cosmetic hair compositions, in particular intended for cleansing and conditioning the hair, which are capable of providing conditioning properties that are not only satisfactory but also persistent with respect to washing, for example persistent after at least five shampooing operations, are also known. These compositions comprise anionic surfactants, non-ionic surfactants, amphoteric surfactants, and also cationic or amphoteric polymers, and organosilanes.

The hair compositions described in the prior art make it possible to cleanse the hair and provide adequate conditioning properties, in particular in terms of disentangling and of softness to the touch. These properties may even be persistent with respect to several shampooing operations. However, these compositions do not make it possible to give the head of hair good styling properties, such as ease of hair shaping and manageability, and also volume and body.

In point of fact, individuals with fine hair or hair that is becoming thinner often use “volumizing” shampoos in order to give their head of hair volume, density and body. The current shampoos do not have optimal properties, and can be further improved with regard to their provision of volume; in addition, the hair care ingredients that they contain can make the hair too lank, thus compromising the volume.

Styling polymers which promote hair shaping are moreover known, but the result in terms of feel of the keratin fibre can prove to be not very satisfactory. In addition, most styling polymers are water-soluble and may be removed by the rinsing water. They cannot therefore be satisfactorily formulated in a rinse-out composition of shampoo type.

The aim of the present invention is to overcome the drawbacks of the prior art, and to provide a cosmetic composition, in particular for cleansing the hair, which makes it possible to obtain, on the one hand, a high cosmetic level, in particular in terms of smoothing (feel and visual aspect), disentangling, suppleness, softness and/or sheen, and on the other hand, good properties of volume, body, density and manageability of the head of hair.

The applicant has discovered, surprisingly, that this objective can be achieved by virtue of a composition comprising a particular combination of anionic, non-ionic and amphoteric surfactants, organosilanes and one or more particular cationic polymers.

One subject of the present invention is thus a cosmetic composition, in particular a cosmetic hair composition, comprising:
one or more anionic surfactants,
one or more amphoteric surfactants,
one or more non-ionic surfactants,
at least two organosilanes, different from one another, and
one or more associative cationic polymers.

It has been noted that the composition according to the invention has good usage qualities, in particular a good foaming capacity, in particular a good foam initiation and/or an abundant foam which is persistent.

The composition according to the invention also makes it possible to give the hair strong cosmetic benefits, in particular in terms of smoothing, disentangling, suppleness, softness and sheen, and also styling properties suitable for fine hair (density, manageability, body, volume).

Advantageously, the competition according to the invention may be clear, transparent.

The clarity of the composition according to the invention can be characterized by the measurement of its turbidity, by turbidemitry (in NTU units). In the context of the present invention, the turbidity measurements were carried out with a turbidimeter, model HI 88713-ISO from the company Hanna Instruments.

Preferably, the turbidity of the compositions according to the invention, measured at ambient temperature (25° C.) and atmospheric pressure, is less than 400 NTU units, more preferentially between 1 and 200 NTU units, even better still between 2 and 100 NTU units, or even between 3 and 50 NTU units.

In the following text, and unless otherwise indicated, the limits of a range of values are included in that range, especially in the expressions “between” and “ranging from . . . to . . . ”.

Moreover, the expressions “at least one” and “at least” used in the present description are equivalent to the expressions “one or more” and “greater than or equal to”, respectively.

Anionic Surfactants

The composition according to the invention comprises at least one anionic surfactant.

The term “anionic surfactant” means a surfactant comprising, as ionic or ionizable groups, only anionic groups.

In the present description, a species is termed “anionic” when it bears at least one permanent negative charge or when it can be ionized into a negatively charged species, under the conditions of use of the composition of the invention (for example the medium or the pH), not comprising any cationic charge.

The anionic surfactants may be sulfate, sulfonate and/or carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO—) and may optionally also comprise one or more sulfate and/or sulfonate functions;
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise any carboxylate or sulfonate functions.

The carboxylic anionic surfactants that may be used thus comprise at least one carboxylic or carboxylate function (—COOH or —COO—).

They can be chosen from the following compounds: acylglycinates, acyllactylates, acylsarcosinates, acylglutamates, alkyl ether carboxylic acids, alkyl(C₆₋₃₀ aryl) ether carboxylic acids, alkyl-D-galactoside-uronic acids, alkylamido ether carboxylic acids; and also the salts of these compounds; the alkyl and/or acyl groups of these compounds comprising from 6 to 30 carbon atoms, in particular from 12 to 28, even better still from 14 to 24 or even from 16 to 22 carbon atoms; the aryl group preferably denoting a phenyl or benzyl group; these compounds possibly being polyoxyalkylenated, in particular polyoxyethylenated, and then preferably comprising from 1 to 50 ethylene oxide units, better still from 2 to 10 ethylene oxide units;

Use may also be made of the C₆-C₂₄ alkyl monoesters of polyglycoside-polycarboxylic acids, such as C₆-C₂₄ alkyl polyglycoside-citrates, C₆-C₂₄ alkyl polyglycosidetartrates and C₆-C₂₄ alkyl polyglycoside-sulfosuccinates, and salts thereof.

Among the above carboxylic surfactants, mention may be made most particularly of polyoxyalkylenated alkyl(amido) ether carboxylic acids and salts thereof, in particular those including from 2 to 50 alkylene oxide and in particular ethylene oxide groups, such as the compounds sold by the company Kao under the Akypo names. The polyoxyalkylenated alkyl(amido) ether carboxylic acids that may be used are preferably chosen from those of formula (1):

R_{1′}—(OC₂H₄)_{n′}-OCH₂COOA (1)

in which:
R_{1′} represents a linear or branched C₆-C₂₄ alkyl or alkenyl radical, a (C₈-C₉)alkylphenyl radical, an R_{2′}CONH—CH₂-CH₂— radical with R_{2′} denoting a linear or branched C₉-C₂₁ alkyl or alkenyl radical; preferably R_{1′} is a C₈-C₂₀, preferably C₈-C₁₈, alkyl radical,
n′ is an integer or decimal number (average value) ranging from 2 to 24 and preferably from 2 to 10,
A denotes H, ammonium, Na, K, Li, Mg or a monoethanolamine or triethanolamine residue.

Use may also be made of mixtures of compounds of formula (1), in particular mixtures of compounds bearing different groups R1′.

The polyoxyalkylenated alkyl(amido)ether carboxylic acids that are particularly preferred are those of formula (1) in which:
R_{1′} denotes a linear or branched C₈-C₂₂, in particular C₁₀-C₁₆ or even C₁₂-C₁₄ alkyl radical, or alternatively a (C₈-C₉)alkylphenyl radical;
A denotes a hydrogen or sodium atom, and
n′ ranges from 2 to 20, preferably from 2 to 10.

Even more preferentially, use is made of the compounds of formula (1) in which R_{1′} denotes a C₁₂-C₁₄ alkyl, cocoyl, oleyl, nonylphenyl or octylphenyl radical, A denotes a hydrogen or sodium atom and n′ ranges from 2 to 10.

Among the commercial products that may preferably be used are the products sold by the company Kao under the names:

Akypo® NP 70 (R₁=nonylphenyl, n=7, A=H)

Akypo® NP 40 (R₁=nonylphenyl, n=4, A=H)

Akypo® OP 40 (R₁=octylphenyl, n=4, A=H)

Akypo® OP 80 (R₁=octylphenyl, n=8, A=H)

Akypo® OP 190 (R₁=octylphenyl, n=19, A=H)

Akypo® RLM 38 (R₁=(C₁₂-C₁₄)alkyl, n=4, A=H)

Akypo® RLM 38 NV (R₁=(C₁₂-C₁₄)alkyl, n=4, A=Na)

Akypo® RLM 45 CA (R₁=(C₁₂-C₁₄)alkyl, n=4.5, A=H)

Akypo® RLM 45 NV (R₁=(C₁₂-C₁₄)alkyl, n=4.5, A=Na)

Akypo® RLM 100 (R₁=(C₁₂-C₁₄)alkyl, n=10, A=H)

Akypo® RLM 100 NV (R₁=(C₁₂-C₁₄)alkyl, n=10, A=Na)

Akypo® RLM 130 (R₁=(C₁₂-C₁₄)alkyl, n=13, A=H)

Akypo® RLM 160 NV (R₁=(C₁₂-C₁₄)alkyl, n=16, A=Na),

or by the company Sandoz under the names:

Sandopan DTC-Acid (R₁=(C₁₃)alkyl, n=6, A=H)

Sandopan DTC (R₁=(C₁₃)alkyl, n=6, A=Na)

Sandopan LS 24 (R₁=(C₁₂-C₁₄)alkyl, n=12, A=Na)

Sandopan JA 36 (R₁=(C₁₃)alkyl, n=18, A=H),

and more particularly the products sold under the following names:

Akypo® RLM 45 (INCI: Laureth-5 carboxylic acid)

Akypo® RLM 100

Akypo® RLM 38.

Preferentially, the carboxylic anionic surfactants are chosen, alone or as a mixture, from:
acylglutamates, in particular of C₆-C₂₄ or even C₁₂-C₂₀, such as stearoylglutamates, and in particular disodium stearoylglutamate;
acylsarcosinates, in particular of C₆-C₂₄ or even C₁₂-C₂₀, such as palmitoylsarcosinates, and in particular sodium palmitoylsarcosinate;
acyllactylates, in particular of C₁₂-C₂₈ or even C₁₄-C₂₄, such as behenoyllactylates, and in particular sodium behenoyllactylate;
C₆-C₂₄ and especially C₁₂-C₂₀ acylglycinates;
(C₆-C₂₄)alkyl ether carboxylates, and in particular (C₁₂-C₂₀)alkyl ether carboxylates; in particular those comprising from 2 to 50 ethylene oxide groups;
polyoxyalkylenated (C₆-C₂₄)alkylamido ether carboxylic acids, in particular those comprising from 2 to 50 ethylene oxide groups;
in particular in acid form or in the form of alkali metal or alkaline-earth metal, ammonium or amino alcohol salts.

Preferably, polyoxyalkylenated (C₆-C₂₄)alkyl ether carboxylic acids and salts thereof are used.

The sulfonate anionic surfactants that may be used comprise at least one sulfonate function (—SO₃H or —SO₃⁻). They may be chosen from the following compounds: alkylsulfonates, alkyl ether sulfonates, alkylamidesulfonates, alkylarylsulfonates, α-olefin sulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds;

the alkyl groups of these compounds comprising from 6 to 30 carbon atoms, especially from 12 to 28, even better still from 14 to 24 or even from 16 to 22, carbon atoms; the aryl group preferably denoting a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Preferentially, the sulfonate anionic surfactants are chosen, alone or as a mixture, from:
C₆-C₂₄ and especially C₁₂-C₂₀ olefin sulfonates;
C₆-C₂₄ and especially C₁₂-C₂₀ alkylsulfosuccinates, especially laurylsulfosuccinates;
C₆-C₂₄ and especially C₁₂-C₂₀ alkyl ether sulfosuccinates;
(C₆-C₂₄)acylisethionates and preferably (C₁₂-C₁₈)acylisethionates; in particular in the form of alkali metal or alkaline-earth metal, ammonium or amino alcohol salts.

The sulfate anionic surfactants that may be used comprise at least one sulfate function (—OSO₃H or —OSO₃⁻).

They may be chosen from the following compounds: alkyl sulfates, alkyl ether sulfates, alkylamido ether sulfates, alkylaryl polyether sulfates, monoglyceride sulfates; and also the salts of these compounds;

the alkyl groups of these compounds comprising from 6 to 30 carbon atoms, especially from 12 to 28, even better still from 14 to 24 or even from 16 to 22, carbon atoms; the aryl group preferably denoting a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Preferentially, the sulfate anionic surfactants are chosen, alone or as a mixture, from:
alkyl sulfates, in particular of C₆-C₂₄ or even C₁₂-C₂₀,
alkyl ether sulfates, in particular of C₆-C₂₄ or even C₁₂-C₂₀, preferably comprising from 2 to 20 ethylene oxide units;

in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the anionic surfactants are chosen, alone or as a mixture, from:
C₆-C₂₄ and especially C₁₂-C₂₀ alkyl sulfates;
C₆-C₂₄ and especially C₁₂-C₂₀ alkyl ether sulfates; preferably comprising from 2 to 20 ethylene oxide units;
C₆-C₂₄ and in particular C₁₂-C₂₀ alkylsulfosuccinates, in particular laurylsulfosuccinates;
C₆-C₂₄ and especially C₁₂-C₂₀ olefin sulfonates;
C₆-C₂₄ and especially C₁₂-C₂₀ alkyl ether sulfosuccinates;
(C₆-C₂₄)acylisethionates and preferably (C₁₂-C₁₈)acylisethionates;
C₆-C₂₄ and in particular C₁₂-C₂₀ acylsarcosinates; in particular palmitoylsarcosinates;
(C₆-C₂₄)alkyl ether carboxylates, preferably (C₁₂-C₂₀)alkyl ether carboxylates; in particular those comprising from 2 to 50 ethylene oxide groups;
polyoxyalkylenated (C₆-C₂₄)alkylamido ether carboxylic acids and salts thereof, in particular those comprising from 2 to 50 alkylene oxide and in particular ethylene oxide groups;
C₆-C₂₄ and especially C₁₂-C₂₀ acylglutamates;
C₆-C₂₄ and especially C₁₂-C₂₀ acylglycinates;

in particular in acid form or in the form of alkali metal or alkaline-earth metal, ammonium or amino alcohol salts.

Preferentially, the composition comprises one or more sulfate anionic surfactants, preferably one or more C₆-C₂₄, in particular C₁₂-C₂₀, alkyl sulfates, and/or one or more C₆-C₂₄, in particular C₁₂-C₂₀, alkyl ether sulfates; preferably comprising from 2 to 20 ethylene oxide units; particularly in the form of alkali metal or alkaline-earth metal, ammonium or amino alcohol salts.

In one particular embodiment of the invention, the composition comprises one or more carboxylic anionic surfactants, and optionally one or more sulfate anionic surfactants.

Preferably, it comprises one or more anionic surfactants of the type polyoxyalkylenated alkyl(amido) ether carboxylic acid of formula (1) above; particularly in acid form or in the form of alkali metal or alkaline-earth metal, ammonium or amino alcohol salts; preferentially in acid form.

Even better still, the composition may comprise:
one or more carboxylic anionic surfactants; preferably of the type polyoxyalkylenated alkyl(amido) ether carboxylic acid of formula (1) above; particularly in acid form or in the form of alkali metal or alkaline-earth metal, ammonium or amino alcohol salts; and
one or more sulfate anionic surfactants; preferably of C₆-C₂₄, in particular C₁₂-C₂₀, alkyl sulfate type, and/or of C₆-C₂₄, in particular C₁₂-C₂₀, alkyl ether sulfate type; preferably comprising from 2 to 20 ethylene oxide units; particularly in the form of alkali metal or alkaline-earth metal, ammonium or amino alcohol salts.

The anionic surfactant(s) are preferably present in the composition according to the invention in a total amount ranging from 1% to 35% by weight, in particular from 3% to 25% by weight and better still from 5% to 22% by weight relative to the total weight of the composition.

In one preferred embodiment, the composition according to the invention comprises
one or more carboxylic anionic surfactants of the type polyoxyalkylenated alkyl(amido) ether carboxylic acid of formula (1) above; particularly in acid form or in the form of alkali metal or alkaline-earth metal, ammonium or amino alcohol salts, in a content preferably ranging from 1% to 15% by weight, in particular from 1.5% to 10% by weight, better still from 2% to 5% by weight relative to the total weight of the composition.
and optionally one or more C₆-C₂₄, in particular C₁₂-C₂₀, alkyl sulfate anionic surfactants, and/or one or more C₆-C₂₄, in particular C₁₂-C₂₀, alkyl ether sulfate surfactants; preferably comprising from 2 to 20 ethylene oxide units; particularly in the form of alkali metal or alkaline-earth metal salts, ammonium salts or amino alcohol salts; in a content preferably ranging from 1% to 15% by weight, in particular from 2% to 12% by weight, better still from 5% to 10% by weight, relative to the total weight of the composition.

Amphoteric Surfactants

The composition according to the invention also comprises one or more amphoteric surfactants.

In particular, the amphoteric surfactants are non-silicone surfactants. They may especially be optionally quaternized secondary or tertiary aliphatic amine derivatives, in which the aliphatic group is a linear or branched chain containing from 8 to 22 carbon atoms, said amine derivatives containing at least one anionic group, for instance a carboxylate, sulfonate, sulfate, phosphate or phosphonate group.

Mention may particularly be made of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines; alone or as a mixture, in particular with the compounds mentioned below.

Among the optionally quaternized derivatives of secondary or tertiary aliphatic amines that may be used, as defined above, mention may also be made of the compounds having the respective structures (II) and (III) below:

Rₐ—CONHCH₂CH₂—N⁺(R_{b})(R_{c})—CH₂COO⁻,M⁺,X⁻ (II)

in which:
Rₐ represents a C₁₀ to C₃₀ alkyl or alkenyl group derived from an acid RₐCOOH preferably present in hydrolysed coconut kernel oil, or a heptyl, nonyl or undecyl group;
R_{b} represents a β-hydroxyethyl group; and
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

R_{a′}—CONHCH₂CH₂—N(B)(B′) (III)

in which:
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
R_{a′} represents a C₁₀ to C₃₀ alkyl or alkenyl group of an acid R_{a′}—COOH which is preferably present in coconut kernel oil or in hydrolysed linseed oil, or an alkyl group, especially a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (IV):

R_{a″}—NHCH(Y″)—(CH₂)ₙCONH(CH₂)_{n′}—N(R_{d})(Rₑ) (IV)

in which:
Y″ represents the group —COOH, —COOZ″ or —CH₂—CH(OH)SO₃H or the group CH₂CH(OH)SO₃—Z″;
R_{d} and Rₑ represent, independently of one another, a C₁ to C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀ to C₃₀ alkyl or alkenyl group of an acid R_{a″}—COOH which is preferably present in coconut kernel oil or in hydrolysed linseed oil;
n and n′ denote, independently of one another, an integer ranging from 1 to 3.

Mention may be made, among the compounds of formula (IV), of the compound classified in the CTFA dictionary under the name sodium diethylaminopropyl cocoaspartamide and sold by Chimex under the name Chimexane HB.

These compounds may be used alone or as mixtures.

Among the amphoteric surfactants, use is preferably made of (C₈-C₂₀)alkylbetaines such as cocobetaine, (C₈-C₂₀)alkylamido(C₃-C₈)alkylbetaines such as cocamidopropylbetaine, and mixtures thereof, and the compounds of formula (IV) such as the sodium salt of diethylaminopropyl laurylaminosuccinamate (INCI name: sodium diethylaminopropyl cocoaspartamide).

Preferentially, the amphoteric or zwitterionic surfactants are chosen from (C₈-C₂₀)alkylamido (C₃-C₈)alkylbetaines such as cocamidopropyl betaine.

Preferably, the composition according to the invention comprises the amphoteric surfactant(s) in a total content ranging from 0.1% to 20% by weight, preferentially in a content ranging from 1% to 15% by weight and even better still from 2% to 10% by weight relative to the total weight of the composition.

Non-Ionic Surfactants

The cosmetic composition according to the invention also comprises one or more non-ionic surfactants, in particular such as those described in “Handbook of Surfactants” by M. R. Porter, published by Blackie & Son (Glasgow and London), 1991, pages 116-178.

Examples of non-ionic surfactants that may be mentioned include the following compounds, alone or as a mixture:
oxyalkylenated (C₈-C₂₄)alkylphenols;
saturated or unsaturated, linear or branched, oxyalkylenated or glycerolated C₈ to C₄₀ alcohols, preferably comprising one or two fatty chains;
saturated or unsaturated, linear or branched, oxyalkylenated C₈ to C₃₀ fatty acid amides;
esters of saturated or unsaturated, linear or branched, C₈ to C₃₀ acids and of polyethylene glycols;
preferably oxyethylenated esters of saturated or unsaturated, linear or branched, C₈ to C₃₀ acids and of sorbitol;
esters of fatty acids and of sucrose;
(C₈-C₃₀)alkyl(poly)glucosides, (C₈-C₃₀)alkenyl(poly)glucosides, which are optionally oxyalkylenated (0 to 10 oxyalkylene units) and comprise from 1 to 15 glucose units, (C₈-C₃₀)alkyl(poly)glucoside esters;
saturated or unsaturated oxyethylenated plant oils;
condensates of ethylene oxide and/or of propylene oxide;
N—(C₈-C₃₀)alkylglucamine and N—(C₈-C₃₀)acylmethylglucamine derivatives;
amine oxides.

The oxyalkylene units are more particularly oxyethylene or oxypropylene units, or a combination thereof, preferably oxyethylene units.

The number of moles of ethylene oxide and/or of propylene oxide preferably ranges from 1 to 250, more particularly from 2 to 100 and better still from 2 to 50; the number of moles of glycerol ranges in particular from 1 to 50 and better still from 1 to 10.

Advantageously, the non-ionic surfactants according to the invention do not comprise any oxypropylene units.

Preferably, they comprise a number of moles of ethylene oxide ranging from 1 to 250, in particular from 2 to 100 and better still from 2 to 50.

As examples of glycerolated non-ionic surfactants, use is preferably made of monoglycerolated or polyglycerolated C₈ to C₄₀ alcohols, comprising from 1 to 50 mol of glycerol and preferably from 1 to 10 mol of glycerol.

Mention may be made, as examples of compounds of this type, of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol and octadecanol containing 6 mol of glycerol.

Among the glycerolated alcohols, it is more particularly preferred to use the C₈ to C₁₀ alcohol containing 1 mol of glycerol, the C₁₀ to C₁₂ alcohol containing 1 mol of glycerol and the C₁₂ alcohol containing 1.5 mol of glycerol.

The non-ionic surfactant(s) used in the composition according to the invention are preferentially chosen, alone or as a mixture, from:
saturated or unsaturated, linear or branched, oxyethylenated C₈ to C₄₀ alcohols comprising from 1 to 100 mol of ethylene oxide, preferably from 2 to 50 and more particularly from 2 to 40 mol of ethylene oxide and preferably comprising one or two fatty chains;
saturated or unsaturated oxyethylenated plant oils comprising from 1 to 100 and preferably from 2 to 50 mol of ethylene oxide;
(C₈-C₃₀)alkyl(poly)glucosides, which are optionally oxyalkylenated, preferably with 0 to 10 mol of ethylene oxide and comprise 1 to 15 glucose units;
monoglycerolated or polyglycerolated C₈ to C₄₀ alcohols, comprising from 1 to 50 mol of glycerol and preferably from 1 to 10 mol of glycerol;
saturated or unsaturated, linear or branched, oxyalkylenated C₈ to C₃₀ fatty acid amides;
esters of saturated or unsaturated, linear or branched, C₈ to C₃₀ acids and of polyethylene glycols;
preferably oxyethylenated esters of saturated or unsaturated, linear or branched, C₈ to C₃₀ acids and of sorbitol.

More preferentially, the non-ionic surfactant(s) used in the composition according to the invention are chosen, alone or as a mixture, from:
saturated or unsaturated, linear or branched, oxyethylenated C₈ to C₄₀ alcohols comprising from 1 to 100 mol of ethylene oxide, preferably from 2 to 50 and more particularly from 2 to 40 mol of ethylene oxide and comprising one or two fatty chains, in particular at least one C₈-C₂₀, in particular C₁₀-C₁₈, alkyl chain;
preferably oxyethylenated esters of saturated or unsaturated, linear or branched, C₈ to C₃₀ acids and of sorbitol, and
(C₈-C₃₀)alkyl(poly)glucosides, which are optionally oxyalkylenated, preferably comprising from 0 to 10 mol of ethylene oxide and comprising 1 to 15 glucose units.

In one particular embodiment, the composition comprises one or more non-ionic surfactants chosen from the non-ionic surfactants of alkyl(poly)glycoside type, in particular represented by the general formula below: R1O—(R2O)t-(G)v in which:
R1 represents a linear or branched alkyl or alkenyl radical including 6 to 24 carbon atoms and especially 8 to 18 carbon atoms, or an alkylphenyl radical of which the linear or branched alkyl radical comprises 6 to 24 carbon atoms and especially 8 to 18 carbon atoms,
R2 represents an alkylene radical including 2 to 4 carbon atoms,
G represents a sugar unit including 5 to 6 carbon atoms,
t denotes a value ranging from 0 to 10 and preferably from 0 to 4,
v denotes a value ranging from 1 to 15 and preferably from 1 to 4.

Preferably, the alkyl(poly)glycoside surfactants are compounds of the formula described above in which:
R1 denotes a linear or branched, saturated or unsaturated alkyl radical including from 8 to 18 carbon atoms,
R2 represents an alkylene radical including 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranges from 1 to 15 and preferably from 1 to 4; the mean degree of polymerization more particularly is between 1 and 2.

The glucoside bonds between the sugar units are generally of 1,6 or 1,4 type and preferably of 1,4 type. Preferably, the alkyl(poly)glycoside surfactant is an alkyl (poly)glucoside surfactant. C₈/C₁₆ alkyl (poly)glucosides 1,4, and in particular decyl glucosides and caprylyl/capryl glucosides, are most particularly preferred.

Among the commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or alternatively the products sold by the company Chem Y under the name AG10 LK.

Preferably, use is made of C₈/C₁₆-alkyl (poly)glycosides 1,4, in particular as an aqueous 53% solution, such as those sold by Cognis under the reference Plantacare® 818 UP.

Preferentially, the composition comprises one or more non-ionic surfactants chosen from, alone or in combination, (C₆-C₂₄ alkyl) (poly)glycosides, and more particularly (C₈-C₁₈ alkyl) (poly)glycosides.

Preferably, the composition according to the invention comprises the non-ionic surfactant(s) in a content ranging from 0.05% to 15% by weight, preferably from 0.1% to 10% by weight, preferentially from 0.2% to 5% by weight, relative to the total weight of the composition according to the invention.

Organosilanes

The cosmetic composition according to the present invention comprises at least two organosilanes that are different from one another.

For the purposes of the present invention, the term “different from one another” is intended to mean two organosilanes of which the chemical structures are different.

The organosilanes are preferably chosen from the compounds of formula (I) below, oligomers thereof, hydrolysis products thereof and mixtures thereof:

R₁Si(OR₂)_{z}(R₃)ₓ(OH)_{y} (I)

in which:
R₁ is a cyclic or acyclic, linear or branched, saturated or unsaturated C₁ to C₂₂, in particular C₁ to C₂₀, hydrocarbon-based chain, which may be substituted with a group chosen from amine groups NH₂ or NHR, R being a linear or branched C₁ to C₂₀, in particular C₁ to C₆, alkyl, a C₃ to C₄₀ cycloalkyl or a C₆ to C₃₀ aromatic ring; the hydroxyl group (OH); a thiol group; an aryl group (more particularly benzyl), which is possibly substituted with an NH₂ or NHR group; it being possible for R₁ to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, z denotes an integer ranging from 0 to 3, and x denotes an integer ranging from 0 to 2, with z+x+y=3.

The term “oligomer” is intended to mean the polymerization products of the compounds of formula (I) comprising from 2 to 10 silicon atoms.

Preferably, R₁ is a linear or branched, preferably linear, saturated C₁ to C₂₂ and especially C₁ to C₁₂ hydrocarbon-based chain, which may be substituted with an amine group NH₂ or NHR, R being a C₁ to C₂₀ and especially C₁ to C₆, alkyl.

Preferably, R₂ represents an alkyl group comprising from 1 to 4 carbon atoms, better still a linear alkyl group comprising from 1 to 4 carbon atoms, and preferably an ethyl group.

Preferably, z ranges from 1 to 3.

Preferably, y=0.

Preferentially, z=3, and therefore x=y=0.

Preferably, at least one of the organosilanes is chosen from the compounds of formula (Ia), corresponding to the compounds of formula (I) in which R₁ represents a linear alkyl group comprising from 1 to 18 carbon atoms and more particularly from 1 to 12 carbon atoms, or a C₁ to C₆, preferably C₂ to C₄, aminoalkyl group. More particularly, R₁ represents a linear alkyl group comprising from 1 to 6 carbon atoms and more particularly a methyl group.

More preferentially, at least one of the organosilanes is a compound of formula (Ia) in which:
R₁ represents a methyl group,
R₂ represents an ethyl group, and
z=3, and therefore x=y=0.

Preferably, at least one of the organosilanes is chosen from the compounds of formula (Ib), corresponding to the compounds of formula (I) in which R₁ is a linear or branched, saturated or unsaturated C₁ to C₂₂ hydrocarbon-based chain, substituted with an amine group NH₂ or NHR, R being a C₁ to C₂₀, in particular C₁ to C₆, alkyl, a C₃ to C₄₀ cycloalkyl or a C₆ to C₃₀ aromatic ring. In this variant, R₁ preferably represents a C₁ to C₆, and more preferentially C₂ to C₄, aminoalkyl group.

More preferentially, at least one of the organosilanes is a compound of formula (Ib) in which:
R₁ represents an aminopropyl group,
R₂ represents an ethyl group, and
z=3, and therefore x=y=0.

Preferably, the composition according to the invention comprises at least one organosilane of formula (Ia) chosen from methyltriethoxysilane (MTES), octyltriethoxysilane (OTES), dodecyltriethoxysilane, octadecyltriethoxysilane, hexadecyltriethoxysilane, oligomers thereof and mixtures thereof; and at least one organosilane of formula (Ib) chosen from 3-aminopropyltriethoxysilane (APTES), 2-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, N-(2-aminoethylaminomethyl)phenethyltrimethoxysilane, oligomers thereof, and mixtures thereof.

Preferentially, the composition comprises a mixture of methyltriethoxysilane and 3-aminopropyltriethoxysilane. In other words, the organosilane of formula (Ia) is methyltriethoxysilane and the organosilane of formula (Ib) is 3-aminopropyltriethoxysilane.

The total amount of organosilanes present in the composition according to the present invention preferably ranges from 0.05% to 15% by weight, preferentially from 0.1% to 10% by weight, and even better still from 0.5% to 5% by weight, relative to the total weight of the composition.

The total amount of organosilane(s) of formula (Ia) present in the composition according to the present invention preferably can range from 0.01% to 10% by weight, more preferentially from 0.05% to 5% by weight, and even better still from 0.1% to 3% by weight, relative to the total weight of the composition.

The total amount of organosilane(s) of formula (Ib) present in the composition according to the present invention preferably can range from 0.1% to 10% by weight, preferentially from 0.3% to 8% by weight, and even better still from 0.5% to 5% by weight, relative to the total weight of the composition.

The weight ratio (R) between the total amount of organosilanes of formula (Ia) and the total amount of organosilanes of formula (Ib) present in the composition is preferably less than or equal to 1, preferentially less than or equal to 0.7, better still less than or equal to 0.5 and even better still less than or equal to 0.4. This ratio (R) can preferably be between 0.02 and 0.7, preferentially between 0.05 and 0.5 and even better still between 0.1 and 0.4.

The composition according to the present invention can optionally also comprise one or more acids chosen from organic acids, mineral acids and mixtures thereof, and preferably from organic acids.

This is because the organosilanes used in the composition of the invention, especially those comprising a basic function, may be partially or totally neutralized in order to improve the water solubility thereof. In particular, the neutralizing agent can be chosen from organic acids, mineral acids or mixtures thereof, as defined below. Preferably, the optionally neutralized organosilanes are water-soluble and especially soluble at a concentration of 2%, better still at a concentration of 5% and even better still at a concentration of 10% by weight in water at a temperature of 25° C. and at atmospheric pressure (1 atm). The term “soluble” is intended to mean the formation of a single macroscopic phase.

For the purposes of the present invention, the term “organic acid” is intended to mean an organic acid and/or the associated bases thereof with a pKa of less than or equal to 7, preferably less than or equal to 6, especially ranging from 1 to 6 and preferably from 2 to 5. According to a preferred embodiment, the organic acid(s) are chosen from carboxylic acids, sulfonic acids and mixtures thereofs. In particular, the acid(s) are chosen from saturated or unsaturated carboxylic acids, in particular those comprising from 2 to 8 carbon atoms, which are optionally hydroxylated, and/or optionally comprise several COOH functions.

Preferably, the acid(s) are chosen from lactic acid, propanoic acid, butanoic acid, acetic acid, citric acid, maleic acid, glycolic acid, salicylic acid, malic acid, tartaric acid and mixtures thereofs, and more preferably lactic acid.

The total content of acid(s), in particular organic acid(s), when they are present in the composition according to the present invention, can preferably range from 0.05% to 10% by weight, and preferentially from 0.1% to 5% by weight relative to the total weight of the composition.

Preferably, the composition comprises at least one organosilane of formula (Ia) as previously defined, at least one organosilane of formula (Ib) as previously defined, and at least one organic acid, preferably chosen from carboxylic acids.

Better still, the composition comprises a mixture of methyltriethoxysilane and 3-aminopropyltriethoxysilane, and at least one carboxylic acid, preferentially lactic acid.

Associative Cationic Polymers

The composition according to the invention also comprises one or more associative cationic polymers. These polymers are not silicone polymers, that is to say they do not comprise a silicon (Si) atom.

The term “cationic polymer” means any polymer comprising cationic groups and/or groups that can be ionized to cationic groups.

The term “associative polymer” is intended to mean an amphiphilic polymer that is capable, in an aqueous medium, of reversibly combining with itself or with other molecules. It generally comprises, in its chemical structure, at least one hydrophilic region or group and at least one hydrophobic region or group. In particular, the hydrophobic group may be a fatty hydrocarbon-based chain such as a linear or branched alkyl, linear or branched arylalkyl or linear or branched alkylaryl group comprising at least 8 carbon atoms, preferably 8 to 30 carbon atoms, better still from 12 to 24 carbon atoms.

Among the cationic associative polymers, mention may be made of, alone or as a mixture:
(A) cationic associative polyurethanes, which can be represented by general formula (Ia) below: R—X—(P)n-[L-(Y)m]r-L′-(P′)p-X‘—R’ in which:

R and R′, which may be identical or different, represent a hydrophobic group or a hydrogen atom;

X and X′, which may be identical or different, represent a group comprising an amine function optionally bearing a hydrophobic group, or alternatively the group L″;

L, L′ and L″, which may be identical or different, represent a group derived from a diisocyanate;

P and P′, which may be identical or different, represent a group comprising an amine function optionally bearing a hydrophobic group;

Y represents a hydrophilic group;

r is an integer between 1 and 100 inclusive, preferably between 1 and 50 inclusive and in particular between 1 and 25 inclusive;

n, m and p are each, independently of one another, between 0 and 1000 inclusive; the molecule contains at least one protonated or quaternized amine function and at least one hydrophobic group.

Preferably, the only hydrophobic groups are the groups R and R′ at the chain ends.

One preferred family of cationic associative polyurethanes is the one corresponding to formula (Ia) described above in which:

R and R′ both independently represent a hydrophobic group,

X and X′ each represent a group L″,

n and p are integers which are between 1 and 1000 inclusive, and L, L′, L″, P, P′, Y and m have the meaning indicated above.

Another preferred family of cationic associative polyurethanes is the one corresponding to formula (Ia) above in which:
n=p=0 (the polymers do not comprise units derived from a monomer containing an amine function, incorporated into the polymer during the polycondensation),
the protonated amine functions result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide or a sulfate.

Yet another preferred family of cationic associative polyurethanes is the one corresponding to formula (Ia) above in which:

R and R′ both independently represent a hydrophobic group,

X and X′ both independently represent a group comprising a quaternary amine,

n=p=0, and

L, L′, Y and m have the meaning indicated above.

The number-average molecular weight (Mn) of the cationic associative polyurethanes is preferably between 400 and 500000 inclusive, in particular between 1000 and 400000 inclusive and ideally between 1000 and 300000 inclusive.

The term “hydrophobic group” means a radical or polymer containing a saturated or unsaturated, linear or branched hydrocarbon-based chain, which may contain one or more heteroatoms such as P, O, N or S, or a radical containing a perfluoro or silicone chain. When it denotes a hydrocarbon-based radical, the hydrophobic group comprises at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote a group comprising a tertiary or quaternary amine, X and/or X′ may represent one of the following formulae:

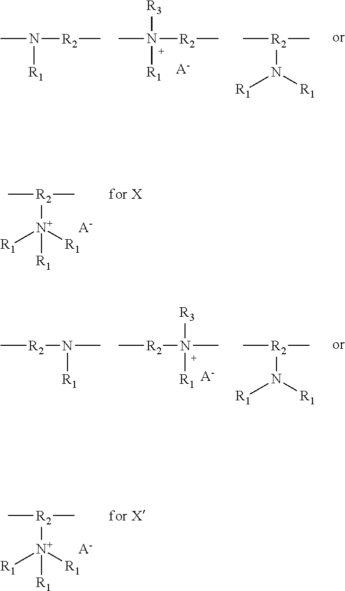

in which:

R₂ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P;

R₁ and R₃, which may be identical or different, denote a linear or branched C₁-C₃₀ alkyl or alkenyl radical or an aryl radical, at least one of the carbon atoms possibly being replaced with a heteroatom chosen from N, S, O and P;

A⁻ is a physiologically acceptable anionic counterion such as a halide, for instance a chloride or bromide, or a mesylate.

The groups L, L′ and L″ represent a group of formula:

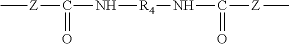

in which:

Z represents —O—, —S— or —NH—; and

R₄ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P.

The groups P and P′ comprising an amine function may represent at least one of the following formulae:

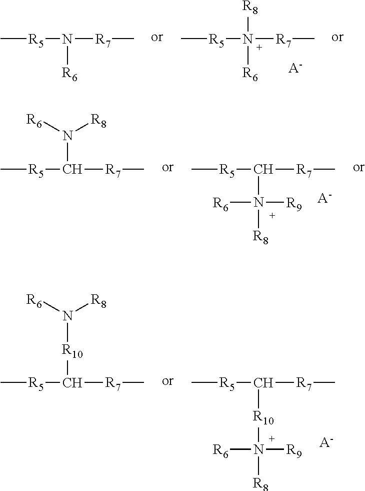

in which:

R₅ and R₇ have the same meanings as R₂ defined above;

R₆, R₈ and R₉ have the same meanings as R₁ and R₃ defined above;

R₁₀ represents a linear or branched, optionally unsaturated alkylene group possibly containing one or more heteroatoms chosen from N, O, S and P;

and A⁻ is a physiologically acceptable anionic counterion such as a halide, for instance chloride or bromide, or mesylate.

As regards the meaning of Y, the term “hydrophilic group” is intended to mean a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, by way of example, of polyethers, sulfonated polyesters, sulfonated polyamides or a mixture of these polymers. Preferentially, the hydrophilic compound is a polyether and in particular a poly(ethylene oxide) or poly(propylene oxide).

The cationic associative polyurethanes of formula (Ia) according to the invention are formed from diisocyanates and from various compounds bearing functions containing a labile hydrogen. The functions containing a labile hydrogen may be alcohol, primary or secondary amine or thiol functions, giving, after reaction with the diisocyanate functions, polyurethanes, polyureas and polythioureas, respectively. The term “polyurethanes” in the present invention encompasses these three types of polymer, namely polyurethanes per se, polyureas and polythioureas, and also copolymers thereof.

A first type of compound involved in the preparation of the polyurethane of formula (Ia) is a compound comprising at least one unit bearing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may comprise more than one unit containing an amine function. It is then a polymer bearing a repetition of the unit containing an amine function.

Compounds of this type may be represented by one of the following formulae: HZ—(P)ₙ—ZH, ou HZ—(P′)ₚ—ZH, in which Z, P, P′, n and p are as defined above.

By way of example, mention may be made of N-methyldiethanolamine, N-tert-butyldiethanolamine and N-sulfoethyldiethanolamine.

The second compound included in the preparation of the polyurethane of formula (Ia) is a diisocyanate corresponding to the formula: O═C═N-R4-N═C═O in which R4 is defined above.

By way of example, mention may be made of methylenediphenyl diisocyanate, methylenecyclohexane diisocyanate, isophorone diisocyanate, tolulene diisocyanate, naphthalene diisocyanate, butane diisocyanate and hexane diisocyanate.

A third compound involved in the preparation of the polyurethane of formula (Ia) is a hydrophobic compound intended to form the terminal hydrophobic groups of the polymer of formula (Ia).

This compound is formed from a hydrophobic group and a function containing a labile hydrogen, for example a hydroxyl, primary or secondary amine, or thiol function.

By way of example, this compound may be a fatty alcohol such as stearyl alcohol, dodecyl alcohol or decyl alcohol. When this compound comprises a polymeric chain, it may be, for example, α-hydroxylated hydrogenated polybutadiene.

The hydrophobic group of the polyurethane of formula (Ia) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine, or thiol functions. This compound may be a polymer terminated at the chain ends with one of these functions containing a labile hydrogen.

By way of example, when it is not a polymer, mention may be made of ethylene glycol, diethylene glycol and propylene glycol.

When it is a hydrophilic polymer, mention may be made, by way of example, of polyethers, sulfonated polyesters and sulfonated polyamides, or a mixture of these polymers. Preferentially, the hydrophilic compound is a polyether and in particular a poly(ethylene oxide) or poly(propylene oxide).

The hydrophilic group termed Y in formula (Ia) is optional. Specifically, the units containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred;
(B) quaternized cellulose derivatives, and in particular quaternized celluloses modified with groups comprising at least one fatty chain, such as linear or branched alkyl groups, linear or branched arylalkyl groups, or linear or branched alkylaryl groups, preferably linear or branched alkyl groups, these groups comprising at least 8 carbon atoms, in particular from 8 to 30 carbon atoms, better still from 10 to 24, or even from 10 to 14, carbon atoms; or mixtures thereof.

Preferably, mention may be made of quaternized hydroxyethylcelluloses modified with groups comprising at least one fatty chain, such as linear or branched alkyl groups, linear or branched arylalkyl groups, or linear or branched alkylaryl groups, preferably linear or branched alkyl groups, these groups comprising at least 8 carbon atoms, in particular from 8 to 30 carbon atoms, better still from 10 to 24, or even from 10 to 14, carbon atoms; or mixtures thereof.

Preferentially, mention may be made of the hydroxyethylcelluloses of formula (Ib):

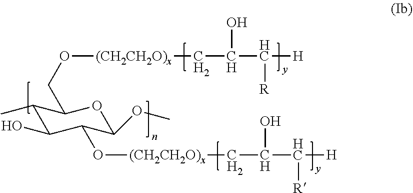

in which:
R represents an ammonium group RaRbRcN⁺-, Q⁻ in which Ra, Rb and Rc, which may be identical or different, represent a hydrogen atom or a linear or branched C₁-C₃₀ alkyl, and Q⁻ represents an anionic counterion such as a halide, for instance a chloride or bromide; preferably an alkyl;
R′ represents an ammonium group R′ₐR′_{b}R′_{c}N⁺—, Q′⁻ in which R′ₐ, R′_{b} and R′_{c}, which may be identical or different, represent a hydrogen atom or a linear or branched C₁-C₃₀ alkyl, and Q′⁻ represents an anionic counterion such as a halide, for instance a chloride or bromide; preferably an alkyl;

it being understood that at least one of the Ra, Rb, Rc, R′a, R′b and R′c radicals represents a linear or branched C₈-C₃₀ alkyl;
n, x and y, which may be identical or different, represent an integer between 1 and 10000.

Preferably, in formula (Ib), at least one of the Ra, Rb, Rc, R′a, R′b and R′c radicals represents a linear or branched C₈-C₃₀, better still C₁₀-C₂₄, or even C₁₀-C₁₄, alkyl; mention may in particular be made of the dodecyl radical (C₁₂). Preferably, the other radical(s) represent a linear or branched C₁-C₄ alkyl, in particular methyl.

Preferably, in formula (Ib), just one of the Ra, Rb, Rc, R′a, R′b and R′c radicals represents a linear or branched C₈-C₃₀, better still C₁₀-C₂₄, or even C₁₀-C₁₄, alkyl; mention may in particular be made of the dodecyl radical (C₁₂). Preferably, the other radicals represent a linear or branched C₁-C₄ alkyl, in particular methyl.

Even better still, R may be a group chosen from —N⁺(CH₃)₃, Q′⁻ and —N⁺(C₁₂H₂₅)(CH₃)₂, Q′⁻, preferably an —N⁺(CH₃)₃, Q′⁻ group.

Even better still, R′ may be an —N⁺(C₁₂H₂₅)(CH₃)₂, Q′⁻ group.

The aryl radicals preferably denote phenyl, benzyl, naphthyl or anthryl groups.

Mention may particularly made of the following polymers having the INCI names:
Polyquaternium-24, such as the product Quatrisoft LM 200@, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by the company Croda.

Mention may also be made of the hydroxyethylcelluloses of formula (Ib) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from the company Amerchol/Dow Chemical.

More particularly, the polymers of formula (Ib) are those of which the viscosity is between 2000 and 3000 cPs inclusive, preferentially between 2700 and 2800 cPs. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs;
(C) cationic polyvinyllactams, in particular those comprising:
a) at least one monomer of vinyllactam or alkylvinyllactam type;
b) at least one monomer of structure (Ic) or (IIc) below:

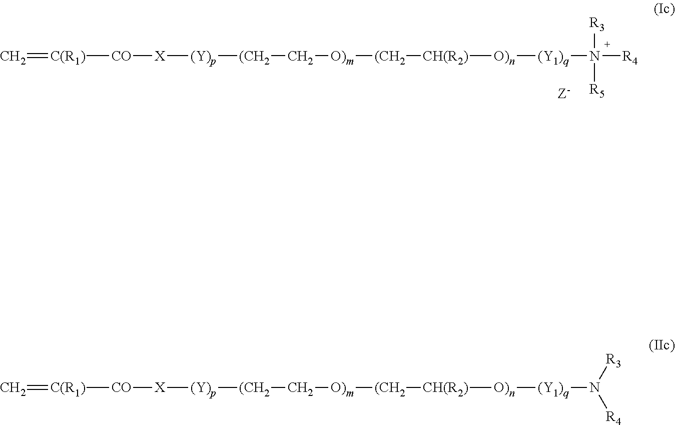

in which:
X denotes an oxygen atom or an NR₆ radical,
R₁ and R₆ denote, independently of one another, a hydrogen atom or a linear or branched C₁-C₅ alkyl radical,
R₂ denotes a linear or branched C₁-C₄ alkyl radical,
R₃, R₄ and R₅ denote, independently of one another, a hydrogen atom, a linear or branched C₁-C₃₀ alkyl radical or a radical of formula (IIIc):

—(Y₂)ᵣ—(CH₂—CH(R₇)—O)ₓ—R₈ (IIIc)
Y, Y₁ and Y₂ denote, independently of one another, a linear or branched C₂-C₁₆ alkylene radical,
R₇ denotes a hydrogen atom, or a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of one another, 0 or 1,
m and n denote, independently of one another, an integer ranging from 0 to 100 inclusive,
x denotes an integer ranging from 1 to 100 inclusive,
Z denotes an anionic counterion of an organic or mineral acid, such as a halide, for instance chloride or bromide, or mesylate;
with the proviso that:
at least one of the substituents R₃, R₄, R₅ or R₈ denotes a linear or branched C₉-C₃₀ alkyl radical,
if m and/or n is other than zero, then q is equal to 1,
if m=n=0, then p or q equals 0.

The cationic poly(vinyllactam) polymers according to the invention may be cross-linked or noncrosslinked and may also be block polymers.

Preferably, the counterion Z⁻ of the monomers of formula (Ic) is chosen from halide ions, phosphate ions, the methosulfate ion and the tosylate ion.

Preferably, R₃, R₄ and R₅ denote, independently of one another, a hydrogen atom or a linear or branched C₁-C₃₀ alkyl radical.

More preferentially, the monomer b) is a monomer of formula (Ic) for which, preferentially, m and n are equal to zero.

The vinyllactam or alkylvinyllactam monomer is preferably a compound of structure (IVc):

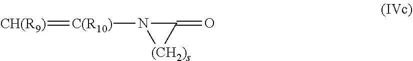

in which:
s denotes an integer ranging from 3 to 6,
R₉ denotes a hydrogen atom or a linear or branched C₁-C₅ alkyl radical,
R₁₀ denotes a hydrogen atom or a linear or branched C₁-C₅ alkyl radical, with the proviso that at least one of the radicals R₉ and R₁₀ denotes a hydrogen atom;

Even more preferentially, the monomer (IVc) is vinylpyrrolidone.

The cationic poly(vinyllactam) polymers according to the invention may also contain one or more additional monomers, preferably cationic or non-ionic monomers.

As compounds that are particularly preferred, mention may be made of the following terpolymers comprising at least:

a) a monomer of formula (IVc),

b) a monomer of formula (Ic) in which p=1, m=n=q=0, R₃ and R₄ denote, independently of one another, a hydrogen atom or a C₁-C₅ alkyl radical and R₅ denotes a linear or branched C₉-C₂₄ alkyl radical, and

c) a monomer of formula (IIc) in which p=1, m=n=q=0, and R₃ and R₄ denote, independently of one another, a hydrogen atom or a linear or branched C₁-C₅ alkyl radical.

Even more preferentially, terpolymers comprising, by weight, 40% to 95% of monomer (a), 0.1% to 55% of monomer (c) and 0.25% to 50% of monomer (b) will be used. Such polymers are described, in particular, in patent application WO-00/68282.

As cationic poly(vinyllactam) polymers according to the invention, use is in particular made of:
vinylpyrrolidone/dimethylaminopropylmethacrylamide/dodecyldimethylmethacrylamidopropylammonium tosylate terpolymers,
vinylpyrrolidone/dimethylaminopropylmethacrylamide/cocoyldimethylmethacrylamidopropylammonium tosylate terpolymers,
vinylpyrrolidone/dimethylaminopropylmethacrylamide/lauryldimethylmethacrylamidopropylammonium tosylate or chloride terpolymers.

The vinylpyrrolidone/dimethylaminopropylmethacrylamide/lauryldimethylme-thylacrylamidopropylammonium chloride terpolymer is in particular provided by the company ISP under the names Styleze W10® or Styleze W20L® (INCI name Polyquaternium-55).

The weight-average molecular weight (Mw) of the cationic poly(vinyllactam) polymers is preferably between 500 and 20000000, more particularly between 200000 and 2000000 and preferentially between 400000 and 800000;
(D) the cationic polymers obtained by polymerization of a monomer mixture comprising one or more vinyl monomers substituted with one or more amino groups, one or more hydrophobic non-ionic vinyl monomers, and one or more associative vinyl monomers, as described in patent application WO 2004/024779.

Among these polymers, mention may more particularly be made of the products of the polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide unit),
a 30/5 polyethylene glycol/polypropylene glycol allyl ether,
a hydroxy(C₂-C₆ alkyl) methacrylate, and
an ethylene glycol dimethacrylate.

Such a polymer is for example the compound sold by the company Lubrizol under the name Carbopol Aqua CC® which corresponds to the INCI name Polyacrylate-1 crosspolymer.

Preferably, the composition comprises one or more associative cationic polymers chosen from the quaternized cationic polymers (B) derived from cellulose, particularly from the hydroxyethylcelluloses of formula (Ib) and even better still polyquaternium-67.

The composition according to the invention can comprise the associative cationic polymer(s) in a total amount ranging from 0.01% to 8% by weight, in particular from 0.05% to 5% by weight and preferentially from 0.1% to 2% by weight, relative to the total weight of the composition.

Other Ingredients

The composition according to the invention may comprise water or a mixture of water and one or more cosmetically acceptable solvents chosen from C₁-C₄ alcohols, such as ethanol, isopropanol, tert-butanol or n-butanol; polyols such as glycerol, propylene glycol and polyethylene glycols; and mixtures thereof.

Preferably, the composition according to the invention has a total water content of between 40% and 95% by weight, preferably between 50% and 90%, preferentially between 60% and 85% by weight relative to the total weight of the composition.

The pH of the compositions according to the invention generally ranges from 3 to 8, preferably from 3.5 to 7 and better still from 4 to 6.5.

The composition according to the invention may also comprise one or more additives that are conventional in cosmetics, such as natural or synthetic thickeners or viscosity regulators, ceramides, vitamins or provitamins, amphoteric or anionic polymers, non-associative cationic polymers, pH agents, preservatives, dyes, fragrances. Those skilled in the art will take care to select the optional additives and the amount thereof such that they do not harm the properties of the compositions of the present invention.

The present invention also relates to a process for the cosmetic treatment of keratin materials, in particular keratin fibres, in particular human keratin fibres such as the hair, using said composition.

The composition according to the invention may be applied to dry or wet keratin fibres, preferably wet keratin fibres.

The composition according to the present invention is generally applied with a leave-in time that may range from 1 to 15 minutes, preferably from 2 to 10 minutes.

After this optional leave-in time, the keratin fibres are optionally rinsed, for example with water, before being dried or left to dry.

The composition according to the invention can thus be advantageously used as a shampoo.

Finally, the present invention relates to the use of said composition for the cosmetic treatment of keratin materials, in particular keratin fibres, in particular human keratin fibres such as the hair, in particular for conferring conditioning properties on said fibres, in particular volume properties, properties providing body and manageability, and also conditioning.

The examples that follow serve to illustrate the invention without, however, being limiting in nature.

Unless otherwise indicated, the amounts of compounds therein are expressed in % by weight of active material (AM) relative to the total weight of the composition.

### EXAMPLE 1

The compositions according to the invention below are prepared:

| Ingredients | Invention A | Invention B | Invention C |
|---|---|---|---|
| Sodium lauryl ether sulfate 2 EO | 7.0 | 7.0 | 7.0 |
| Laureth-5 carboxylic acid | 4.0 | 4.0 | 4.0 |
| Cocamidopropyl betaine | 6.4 | 6.4 | 6.4 |
| Cocoglucoside | 0.5 | 2.0 | — |
| PPG-5-Ceteth-20 | — | — | 2.0 |
| 3-Aminopropyltriethoxysilane | 0.96 | 1.0 | 0.96 |
| Methyltriethoxysilane | 0.14 | 0.15 | 0.14 |
| Polyquaternium-67 | 0.3 | 0.5 | 0.5 |
| Lactic acid | 0.2 | 0.2 | 0.2 |
| NaCl | 1.2 | 2.2 | 1.2 |
| Preservatives, fragrance | qs | qs | qs |
| pH agents | qs pH 5 | qs pH 5 | qs pH 5 |
| Water | qs 100% | qs 100% | qs 100% |

Transparent compositions are obtained.

When used as a shampoo, the compositions according to the invention result in very good performance in terms of disentangling, suppleness, smoothness to the touch, coating, in particular on wet hair.

### Example 2

Composition A of Example 1 is compared with a comparative composition A′ containing a single organosilane.

| Ingredients | Invention A | Comparative A′ |
|---|---|---|
| Sodium lauryl ether sulfate 2 EO | 7.0 | 7.0 |
| Laureth-5 carboxylic acid | 4.0 | 4.0 |
| Cocamidopropyl betaine | 6.4 | 6.4 |
| Cocoglucoside | 0.5 | 0.5 |
| 3-Aminopropyltriethoxysilane | 0.96 | 1.10 |
| Methyltriethoxysilane | 0.14 | — |
| Polyquaternium-67 | 0.3 | 0.3 |
| Lactic acid | 0.2 | 0.2 |
| NaCl | 1.2 | 1.2 |
| Preservatives, fragrance | qs | qs |
| pH agents | qs pH 5 | qs pH 5 |
| Water | qs 100% | qs 100% |

The shampoo compositions are diluted 4% with water in a 100 ml beaker. They are mixed using a magnetic stirrer to homogenize, then introduced into a pump delivering foam. The foam thus produced is harvested in a beaker and the abundance of the foam at T0 and after 3 minutes of rest (25° C.) is measured (by photography and image analysis).

The abundance of the foam is then determined by the % of pixels corresponding to the foam relative to the total number of pixels: a photo is taken at T0 and T3 minutes. The ratio of the number of pixels of the foam (beaker with foam+void) to the number of pixels of the “measuring cell” (beaker with foam+void)×100 is calculated by image analysis. A “percentage occupation of the volume” by the foam is thus obtained.

The results are given in the table below.

| | Invention A | Comparative A′ |
|---|---|---|
| T0 | 65.0 ± 2.0% | 59.7 ± 1.2% |
| T at 3 minutes | 39.0 ± 2.0% | 33.7 ± 3.1% |

It is noted that the amount of foam generated with the invention is significantly greater, whether at T0 (foam initiation) or after 3 minutes (foam persistence). The formula according to the invention thus has better usage qualities.

### Example 3

Composition B of Example 1 was evaluated in comparison with a commercial shampoo intended for fine hair, comprising a combination of sodium laureth sulfate, laureth-5 carboxylic acid, cocamidopropyl betaine, non-ionic surfactants, polyquaternium-67 and aminopropyltriethoxysilane.

The evaluation is carried out blind, by experts, on a panel of 6 individuals with fine hair of medium sensitivity.

6 g of composition are applied, per ½ head, on previously wetted hair. At the end of the shampooing, the hair is rinsed with water (wet hair evaluation) then dried with a hairdryer (dry hair evaluation).

Each criterion is evaluated on a scale of 0 (lowest level) to 5 (highest level), in increments of 0.5.

The following results are obtained (mean of 6 scores):

| | Invention B | Comparative |
|---|---|---|
| Disentangling (wet hair) | 3.6 | 2.8 |
| Suppleness (wet hair) | 3.3 | 2.4 |
| Smooth feel (wet hair) | 3.6 | 2.7 |
| Coating (wet hair) | 3.6 | 2.8 |
| Suppleness (dry hair) | 3.1 | 2.6 |
| Coating (dry hair) | 3.8 | 2.7 |

It is noted that the composition according to the invention has cosmetic qualities (disentangling, suppleness, smoothness), on wet hair and on dry hair, that are superior to the comparative shampoo.

The quality of the coating contributes to the treatment of the strand of hair and to the care effect. In addition, it makes it possible to obtain better ease of styling.

The composition according to the invention provides better manageability and a greater volume effect, which contributes to satisfying the expected volume and styling benefits.

1.-13. (canceled)

14. A cosmetic composition comprising:
at least one anionic surfactant;
at least one amphoteric surfactant;
at least one non-ionic surfactant;
at least two organosilanes, different from one another; and
at least one associative cationic polymer.

15. The composition of claim 14, further comprising at least one carboxylic surfactant chosen from polyoxyalkylenated alkyl(amido)ether carboxylic acids and salts thereof.

16. The composition of claim 15, wherein the polyoxyalkylenated alkyl(amido)ether carboxylic acids and salts are chosen from compounds of formula (1):
R_{1′}—(OC₂H₄)_{n′}-OCH₂COOA (1)
wherein in formula (1):
R_{1′} is chosen from a linear or branched C₆-C₂₄ alkyl or alkenyl radical, a (C₈-C₉)alkylphenyl radical, an R_{2′}CONH—CH₂—CH₂— radical with R_{2′} denoting a linear or branched C₉-C₂₁ alkyl or alkenyl radical;
n′ is an integer or decimal number (average value) ranging from 2 to 24; and
A is chosen from H, ammonium, Na, K, Li, Mg, a monoethanolamine residue, or triethanolamine residue.

17. The composition of claim 14, further comprising at least one sulfate anionic surfactant.

18. The composition of claim 17, wherein the at least one sulfate anionic surfactant is chosen from C₆-C₂₄ alkyl sulfates or C₆-C₂₄ alkyl ether sulfates; and wherein the at least one sulfate anionic surfactant is in the form of alkali metal, alkaline-earth metal, ammonium salts, or amino alcohol salts.

19. The composition of claim 14, wherein the at least one non-ionic surfactant is chosen from:
oxyalkylenated (C₈-C₂₄)alkylphenols;
saturated or unsaturated, linear, or branched, oxyalkylenated or glycerolated C₈ to C₄₀ alcohols;
saturated or unsaturated, linear or branched, oxyalkylenated C₈ to C₃₀ fatty acid amides;
esters of saturated or unsaturated, linear or branched, C₈ to C₃₀ acids, and of polyethylene glycols;
oxyethylenated esters of saturated or unsaturated, linear or branched, C₈ to C₃₀ acids and of sorbitol;
esters of fatty acids and of sucrose;
(C₈-C₃₀)alkyl(poly)glucosides, (C₈-C₃₀)alkenyl(poly)glucosides, which are optionally oxyalkylenated (0 to 10 oxyalkylene units) and comprise from 1 to 15 glucose units, or (C₈-C₃₀)alkyl(poly)glucoside esters;
saturated or unsaturated oxyethylenated plant oils;
condensates of ethylene oxide and/or of propylene oxide;
N—(C₈-C₃₀)alkylglucamine and N—(C₈-C₃₀)acylmethylglucamine derivatives; or
amine oxides.

20. The composition of claim 14, wherein the at least one non-ionic surfactant is chosen from alkyl (poly)glycosides having the general formula below: R1O—(R2O)t-(G)v,
wherein:
R1 is chosen form a linear or branched alkyl or alkenyl radical including 6 to 24 carbon atoms, or an alkylphenyl radical of which the linear or branched alkyl radical comprises 6 to 24 carbon atoms;
R2 is chosen from an alkylene radical including 2 to 4 carbon atoms;
G represents a sugar unit including 5 to 6 carbon atoms;
t denotes a value ranging from 0 to 10; and
v denotes a value of the degree of polymerization, ranging from 1 to 15.

21. The composition of claim 20, wherein in the formula R1O—(R2O)t-(G)v,
R1 is chosen from a linear or branched, saturated or unsaturated alkyl radical including from 8 to 18 carbon atoms;
R2 is chosen from an alkylene radical including 2 to 4 carbon atoms;
G is glucose, fructose, or galactose;
t denotes a value ranging from 0 to 3; and
the degree of polymerization, i.e. the value of v, optionally ranges from 1 to 15, and the mean degree of polymerization is between 1 and 2.

22. The composition of claim 14, wherein the at least two organosilanes that are different from one another are chosen from compounds of formula (I) below, oligomers thereof, hydrolysis products thereof, or mixtures thereof:
R₁Si(OR₂)_{z}(R₃)ₓ(OH)_{y} (I)
wherein in formula (I):
R₁ is a cyclic or acyclic, linear or branched, saturated or unsaturated C₁ to C₂₂ hydrocarbon-based chain, which may be substituted with a group chosen from amine groups NH₂ or NHR, R being a linear or branched C₁ to C₂₀ alkyl, a C₃ to C₄₀ cycloalkyl, or a C₆ to C₃₀ aromatic ring; the hydroxyl group (OH); a thiol group; an aryl group (more particularly benzyl), which is optionally substituted with an NH₂ or NHR group; optionally, R₁ is interrupted with a heteroatom (O, S or NH) or a carbonyl group (CO);
R₂ and R₃, which may be identical or different, represent a linear or branched alkyl group comprising from 1 to 6 carbon atoms; and
y denotes an integer ranging from 0 to 3, z denotes an integer ranging from 0 to 3, and x denotes an integer ranging from 0 to 2, with z+x+y=3.

23. The composition of claim 19, wherein in the compounds of formula (I), R₁ is chosen from a linear alkyl group comprising from 1 to 18 carbon atoms.

24. The composition of claim 19, wherein in the compounds of formula (I), R₁ is a linear or branched, saturated or unsaturated C₁ to C₂₂ hydrocarbon-based chain, substituted with an amine group NH₂ or NHR, R being a C₁ to C₂₀ alkyl, a C₃ to C₄₀ cycloalkyl, or a C₆ to C₃₀ aromatic ring.

25. The composition of claim 14, wherein the at least one associative cationic polymer is chosen from:
(A) cationic associative polyurethanes, which can be represented by general formula (Ia) below:
R—X—(P)n-[L-(Y)m]r-L′-(P′)p—X′—R′,
wherein in formula (Ia):
R and R′, which may be identical or different, represent a hydrophobic group or a hydrogen atom;
X and X′, which may be identical or different, represent a group comprising an amine function, optionally bearing a hydrophobic group, or alternatively the group L′;
L, L′ and L″, which may be identical or different, represent a group derived from a diisocyanate;
P and P′, which may be identical or different, represent a group comprising an amine function optionally bearing a hydrophobic group;
Y represents a hydrophilic group;
r is an integer between 1 and 100; and
n, m, and p are each, independently of one another, between 0 and 1000;
wherein the compounds of formula (Ia) contain at least one protonated or quaternized amine function and at least one hydrophobic group;
(B) quaternized cellulose derivatives;
(C) cationic polyvinyllactams; and
(D) cationic polymers obtained by polymerization of a monomer mixture comprising at least one vinyl monomer substituted with at least one amino group, at least one hydrophobic non-ionic vinyl monomer, or at least one associative vinyl monomer.

26. The composition of claim 25, wherein the quaternized cellulose derivatives are modified with groups comprising at least one fatty chain, wherein the at least one fatty chain includes at least 8 carbon atoms.

27. The composition of claim 25, wherein the quaternized cellulose derivatives comprise quaternized hydroxyethylcelluloses modified with groups comprising at least one fatty chain including at least 8 carbon atoms.

28. The composition of claim 25, wherein the cationic polyvinyllactams comprise:
a) at least one monomer of vinyllactam or alkylvinyllactam type; and
b) at least one monomer of structure (Ic) or (IIc) below: 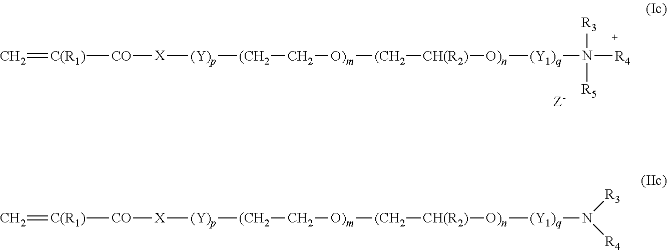
wherein:
X is chosen from an oxygen atom or an NR₆ radical;
R₁ and R₆ is chosen from, independently of one another, a hydrogen atom, or a linear or branched C₁-C₅ alkyl radical;
R₂ is chosen from a linear or branched C₁-C₄ alkyl radical;
R₃, R₄, and R₅ are chosen from, independently of one another, a hydrogen atom, a linear or branched C₁-C₃₀ alkyl radical, or a radical of formula (IIIc):
—(Y₂)ᵣ—(CH₂—CH(R₇)—O)_{c}—R₈ (IIIC)
wherein:
Y, Y₁, and Y₂ are chosen from, independently of one another, a linear or branched C₂-C₁₆ alkylene radical;
R₇ is chosen from a hydrogen atom, a linear or branched C₁-C₄ alkyl radical, or a linear or branched C₁-C₄ hydroxyalkyl radical;
R₈ is chosen from a hydrogen atom, or a linear or branched C₁-C₃₀ alkyl radical;
p, q, and r denote, independently of one another, either the value zero or the value 1;
m and n denote, independently of one another, an integer ranging from 0 to 100 inclusive;
x denotes an integer ranging from 1 to 100 inclusive;
Z denotes an anionic counterion of an organic or mineral acid;
with the proviso that:
at least one of the substituents R₃, R₄, R₅, or R₈ denotes a linear or branched C₉-C₃₀ alkyl radical;
if m and/or n is other than zero, then q is equal to 1, and
if m=n=0, then p or q equals 0.

29. The composition of claim 14, wherein the at least one cationic associative polymer is chosen from the quaternized hydroxyethylcelluloses of formula (Ib): 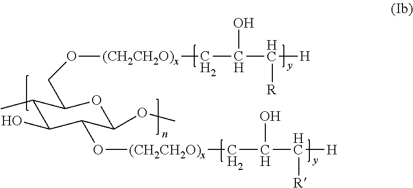
wherein in formula (Ib):
R represents an ammonium group RaRbRcN⁺—, Q⁻, wherein Ra, Rb, and Rc, which may be identical or different, are chosen from a hydrogen atom, or a linear or branched C₁-C₃₀ alkyl, and Q⁻ represents an anionic counterion;
R′ represents an ammonium group R′aR′bR′cN⁺—, Q′⁻, wherein R′a, R′b, and R′c, which may be identical or different, are chosen from a hydrogen atom, or a linear or branched C₁-C₃₀ alkyl, and Q′⁻ represents an anionic counterion;
wherein at least one of the Ra, Rb, Rc, Ra, R′b, and R′c radicals represents a linear or branched C₈-C₃₀ alkyl;
n, x and y, which may be identical or different, represent an integer between 1 and 10 000.

30. The composition of claim 29, wherein in the quaternized hydroxyethylcelluloses of formula (Ib), only one of the Ra, Rb, Rc, R′a, R′b, and R′c radicals represents a linear or branched C₈-C₃₀ alkyl.

31. The composition of claim 14, wherein the at least one associative cationic polymer comprises polyquaternium-67.

32. The composition of claim 14, wherein the at least one associative cationic polymer is present in an amount ranging from 0.01% to 8% by weight, relative to the total weight of the composition.

33. A method of treating keratin materials comprising applying to the keratin materials an effective amount of a composition comprising:
at least one anionic surfactant;
at least one amphoteric surfactant;
at least one non-ionic surfactant;
at least two organosilanes, different from one another; and
at least one associative cationic polymer.
